(19) United States
(12) Plant Patent Application Publication
(10) Pub. No.: US 2003/0182700 P1
(43) Pub. Date: Sep. 25, 2003

(54) Agapanthus orientalis plant named 'Snow Cloud'

(76) Inventor: Vance Hooper, Waitara (NZ)

Correspondence Address: Vance Hooper, Monrovia Nursery Co., Botanical Department, 18331 E Foothill Blvd., P.O. Box 1385, Azusa, CA, 91702, US

(21) Appl. No.: 10/104,537

(22) Filed: Mar. 25, 2002

(51) Int. Cl. A01H 5/00

(52) U.S. Cl. PLT/263

(57) ABSTRACT

A new and distinct cultivar of Lily of the Nile plant named Agapanthus orientalis ‘Snow Cloud’, substantially as shown and described.

### BACKGROUND OF THE INVENTION

This invention relates to a new and distinct selection of Agapanthus orientalis which is native to South Africa and a member of the amaryllis family. Agapanthus orientalis ‘Snow Cloud’ was not the result of a breeding program. The seed was collected in April 1987 at my residence in Waitara, Taranki, New Zealand from a plant of Agapanthus orientalis growing in close proximity to an Agapanthus inapterus. The open pollinated seed was sown and my selection was made in February 1992 at Waitara, Taranki, New Zealand. Tissue culture initiation was begun in 1995 at Lifetech Laboratories Ltd. in Albany, Auckland, New Zealand.

My new selection has been asexually reproduced by tissue culture at Lifetech Laboratories in Auckland, New Zealand since the discovery in 1995. It is unlikely the particular select and favorable attributes of this plant could be conveyed to progeny through sexual reproduction in the next generation. However, through extensive propagation through tissue culture it has been established that the novel exceptional characteristics of this plant are stable and reliably passed on to clonal specimens through asexual reproduction.

### SUMMARY

Agapanthus orientalis cultivar ‘Snow Cloud’ possesses an inflorescence with unusually large numbers of florets. The number of florets is double the number of a typical Agapanthus orientalis inflorescence. This high number of florets is exhibited in the dense, rounded, compact inflorescence that ranges in a diameter of 16 to 20 centimeters.

Perianth color is white with no other color tone evident. The single flowers exhibit a long tubular form typical of the believed pollen parent Agapanthus inapterus. The perianth sections of Agapanthus orientalis are generally six parted. The florets of ‘Snow Cloud’ number six to eight perianth segments, producing a fuller inflorescence.

Normally, seed production is recognized as flowers mature however, in the case of this selection, fruit has not been observed which would be an attribute for landscape use.

Temperatures tolerated have ranged in the low of 10 degree Fahrenheit which is equivalent to an USDA hardiness zone of eight, without exhibiting any damage.

Growth and/or color may vary with the time of year, light conditions, soil, and nutrient conditions. Plants which are grown in shade conditions with greater available nitrogen may exhibit darker green foliage.

### BRIEF DESCRIPTION OF THE DRAWING

The accompanying photographs illustrate the present invention as follows:

FIG. 1 shows a group of ‘Snow Cloud’ plants in flower.

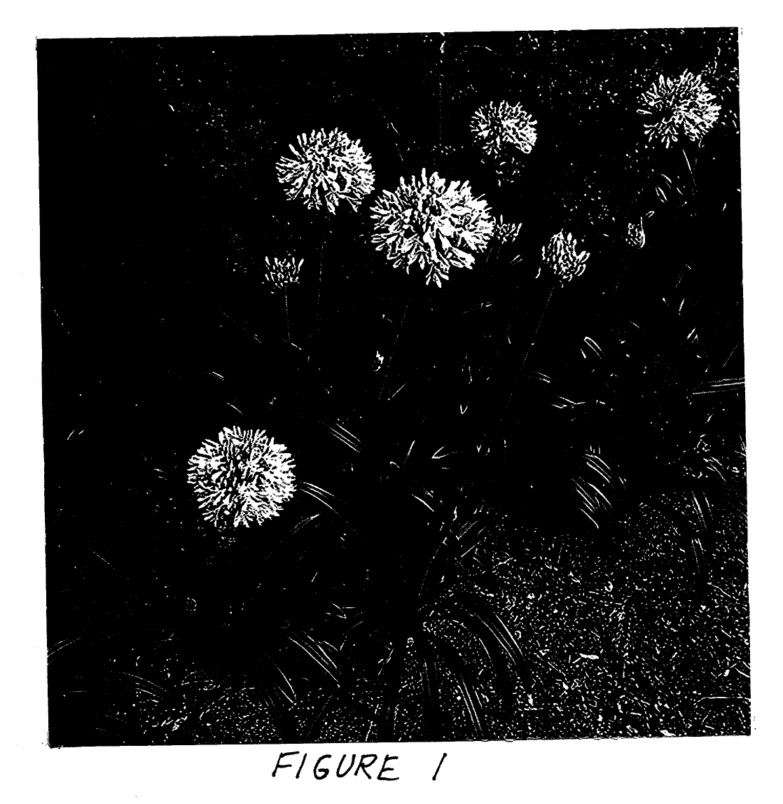

FIG. 2 is a close up of the inflorescence of ‘Snow Cloud’ which exhibits the density of the florets, the uniform white color of the perianth and perianth segments numbering from six to eight.

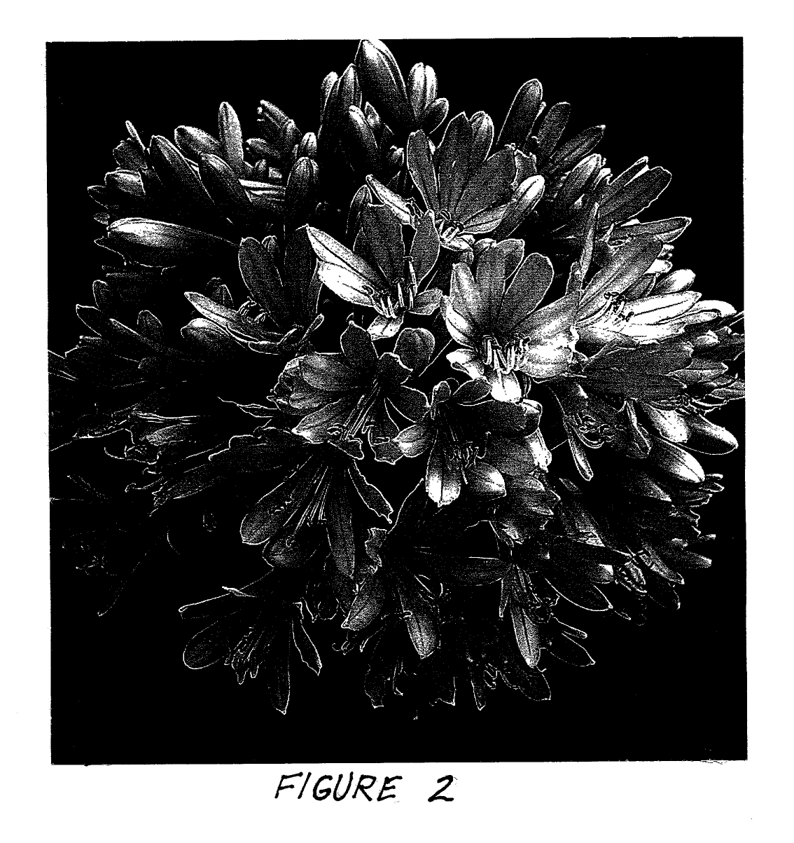

### BOTANICAL DESCRIPTION

Below is a detailed description of Agapanthus orientalis ‘Snow Cloud’. The color terminology is based on The Royal Horticultural Color Charts. Measurements are based on plants grown in Australia and California.

Overall habit: Herbaceous perennial.

Overall size: Maximum height observed from soil surface to top of the inflorescence ranges from 90 to 120 centimeters with a typical range 90 to 100 centimeters. Maximum height from soil surface to upper most of leaves at flowering time ranges approximately 50 centimeters.

Bloom Season in California: Bloom commences in early summer (approximately two to three weeks after Agapanthus ‘Glen Avon’ (U.S. Plant Patent Pending) and continues about three months, therefore, bloom will appear throughout the summer months.

Foliage: Linear, strap-like.

Size:

Length.—From base to the tip of mature foliage, typically longer than 40 centimeters and generally shorter than 60 centimeters.

Width.—Maximum width of mature foliage, typically wider than 2.5 centimeters, generally 3.5 centimeters.

Number.—Leaves per bud at flowering time, typically more than eleven.

Shape of leaf tip.—Obtuse to slightly acute.

Margins.—Smooth, entire.

Color.—Upper leaf surface, Yellow Green Group 146 A. Lower leaf surface, Yellow Green Group 146 A.

Texture.—Slightly fleshy, succulent.

Inflorescence:

Arrangement.—Single umbel, primary and secondary inflorescences.

Width.—Maximum width at time of flowering is typically wider than 16 centimeters, on average 20 centimeters. Secondary inflorescence averaging 18 centimeters.

Height.—Side view at time of flowering is typically ranging from 10 to 20 centimeters, on the average 13 centimeters. Secondary inflorescences averaging 12 centimeters.

Flower:

Scape:

Length.—From soil surface to base of primary inflorescence at the time of flowering is typically ranging from 80 to 90 centimeters, averaging 80 centimeters. The length of scape on secondary inflorescences averages 40 to 58 centimeters.

Thickness.—Typical maximum thickness at the middle point of the scape on a primary inflorescence at time of flowering averaging 1.3 centimeters. Secondary inflorescence scape diameter averages approximately 7 millimeters.

Cross section.—Shape at middle point of scape at time of flowering — Rounded to elliptic.

Angle.—At time of flowering — straight.

Color.—At middle portion of the scape at time of flowering, Yellow-Green Group 146 B.

Florets.—Perianth Shape: Long, tubular, spreading. Number of Perianth Segments: Total six to eight segments, averaging Perianth Color: Inner and outer sides White Group 155D. Color does not change with maturity. Number of Florets per Inflorescence: Approximately 350. Length: Side view from base to tip of floret 3.5 to 4.0 centimeter. Width: Diameter averaging 3.0 centimeter.

Pedicel.—Length: 4.5 to 6 centimeter long. Color: Yellow Green Group 144 A.

Stamens.—Number: Six Arrangement: Adhere to within the throat 1.4 centimeter from base, 2.5 centimeter long from perianth. Anthers: Typically 2 millimeters in length. Yellow Green Group 154 B. Pollen: Yellow Green Group 154 B.

Pistil.—Length of Style: Typically 20 millimeters long. Fruit: Not observed. Disease Resistance: As is typical of this species, the ‘Snow Cloud’ cultivar exhibited a strong disease resistance under normal growing conditions.

I claim:

1. A new and distinct Agapanthus orientalis plant named ‘Snow Cloud’ substantially as shown and described which differs distinctively from other Agapanthus orientalis plants by its unique combination of single white florets, dense inflorescences that produce twice the average number of florets and a prolonged flowering period due to the production of secondary flowering spikes.
